(19) United States
(12) Patent Application Publication
(10) Pub. No.: US 2025/0249285 A1
(43) Pub. Date: Aug. 7, 2025

(54) NEURAL NETWORK-BASED RADIATION DOSE DETERMINATION

(71) Applicant: Siemens Healthineers International AG, Steinhausen (CH)

(72) Inventor: Martin Kraus, Furth (DE)

(21) Appl. No.: 18/435,541

(22) Filed: Feb. 7, 2024

(51) Int. Cl. A61N 5/10; G16H 10/60; G16H 30/40

(52) U.S. Cl. CPC A61N 5/1031; A61N 5/1039; G16H 10/60; G16H 30/40

(57) ABSTRACT

A neural network (such as a transformer neural network) configured to determine radiation doses can be trained using a training corpus that comprises a plurality of different resultant radiation doses (such as, but not limited to, sparsely-written resultant radiation doses) and a plurality of different input items that each correspond to a particular one of the plurality of different resultant radiation doses. Those input items can comprise at least one, two, three, or each of a patient image (such as, but not limited to, computed tomography imagery and/or Digital Imaging and Communications in Medicine-compatible imagery), a fluence map, radiation treatment platform geometry information, and/or target dose volume information (such as, but not limited to, sparsely-read target dose volume information). A radiation dose for a patient can be generated by providing patient image information as input to a trained neural network and outputting a determined radiation dose for the patient.

### TECHNICAL FIELD

These teachings relate generally to treating a patient's planning target volume with energy pursuant to an energy-based treatment plan and more particularly to determining a radiation dose.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Calculating a dose deposition in a patient's body typically helps serve to determine a suitable radiation treatment plan and/or to verify that an optimized existing plan is safe and effective. The various available known approaches in these regards differ in their calculation speed as well as their corresponding accuracy, with no single approach necessarily meeting the needs of all application settings.

### BRIEF DESCRIPTION OF THE DRAWINGS

The above needs are at least partially met through provision of the neural network-based radiation dose determination described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a flow diagram as configured in accordance with various embodiments of these teachings;

FIG. 4 comprises a schematic representation as configured in accordance with various embodiments of these teachings; and

FIG. 5 comprises a schematic representation as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments, a neural network (such as a transformer neural network) configured to determine radiation doses can be trained using a training corpus that comprises a plurality of different resultant radiation doses (such as, but not limited to, sparsely-written resultant radiation doses) and a plurality of different input items that each correspond to a particular one of the plurality of different resultant radiation doses. Those input items can comprise at least one, two, three, or each of a patient image (such as, but not limited to, computed tomography imagery and/or Digital Imaging and Communications in Medicine-compatible imagery), a fluence map, radiation treatment platform geometry information, and/or target dose volume information (such as, but not limited to, sparsely-read target dose volume information).

These teachings can further support determining a radiation dose for a patient by accessing patient image information for the patient, inputting the patient image information (for example, by selecting a read location) (wherein the patient image information may comprise image information for a plurality of different positions) to a neural network that has been trained using the aforementioned training corpus, and outputting from that neural network (for example, by selecting a write location) a determined radiation dose for the patient. These teachings will accommodate so determining a radiation dose for the patient prior to optimizing a radiation treatment plan for the patient and/or subsequent to optimizing a radiation treatment plan for the patient.

So configured, these teachings strike a useful and beneficial balance between accuracy and speed when determining a radiation dose.

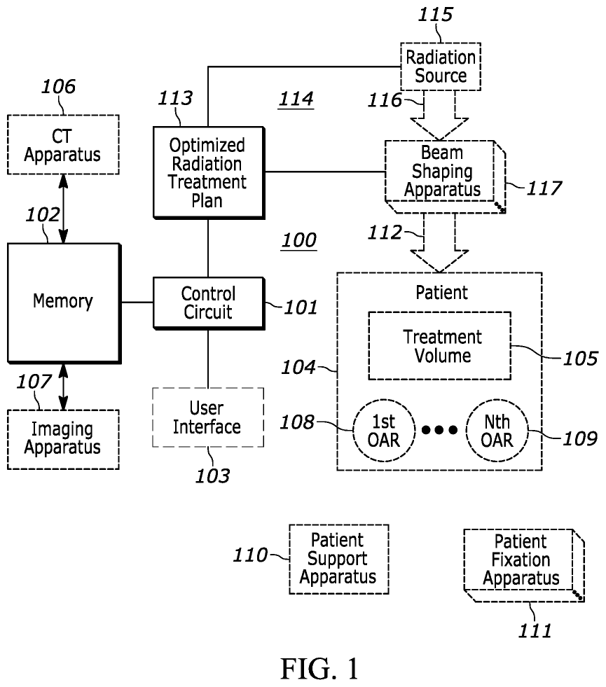

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be understood that a reference to a “control circuit” in the singular may reference a literal single discrete such device or may reference a plurality of discrete control circuits that can be viewed in the aggregate as a “control circuit.” Accordingly, references to a “control circuit” will be understood to alternatively include either a single such device or a plurality of such devices in the absence of any language to the contrary.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as the training corpus described herein, optimization information for a particular patient, and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

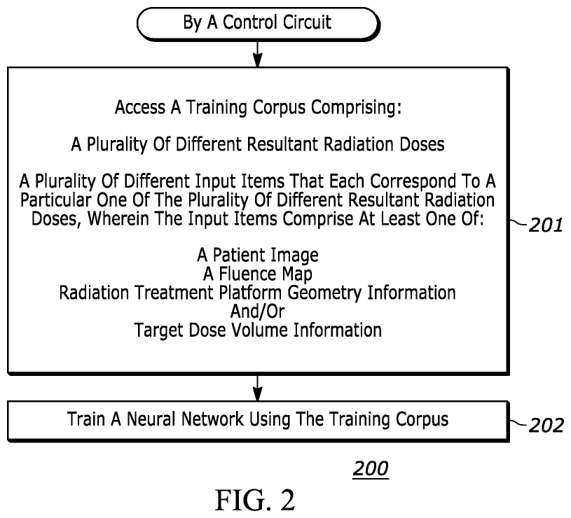

Referring now to FIG. 2, a process 200 that can be carried out, for example, in conjunction with the above-described application setting (and more particularly via the aforementioned control circuit 101) will be described. By one approach, this process 200 can serve to facilitate generating an optimized radiation treatment plan 113 to thereby facilitate treating a particular patient with therapeutic radiation using a particular radiation treatment platform per that optimized radiation treatment plan.

In the following example, the control circuit 101 is configured as a neural network that is configured to determine radiation doses. Neural networks are known in the art and comprise a computational model having a number of interconnected nodes, called neurons, that are organized in layers. Each neuron receives input signals, processes them, and generates an output signal. The strength of the connections between neurons, known as weights, determines the impact of each input on the output. Through a process called training, the network learns to adjust these weights to improve its performance on a given task.

There are various neural networks known in the art. For the sake of an illustrative example, and without intending to suggest any limitations in these regards, it will be presumed here that the neural network comprises a transformer neural network. A transformer neural network is a type of deep learning model that, unlike traditional recurrent neural networks that process input sequentially, relies on a self-attention mechanism to simultaneously capture dependencies between data items. This allows transformer neural networks to efficiently handle long-range dependencies and capture contextual information effectively. The transformer architecture consists of an encoder and a decoder, both composed of multiple layers of self-attention and feed-forward neural networks. The encoder processes the input sequence, while the decoder generates the output sequence.

At block 201, this process 200 provides for accessing a training corpus that includes a plurality of different resultant radiation doses as well as a plurality of different input items that each correspond to a particular one of the plurality of different resultant radiation doses.

By one approach, at least some of the plurality of different resultant radiation doses each comprises sparsely-written resultant radiation doses. Sparsely-written data refers to a type of data where the available information is incomplete or sparse, meaning there are missing values or gaps in the data. In this context, “sparse” does not refer to the density of the data points, but rather to the lack of information or observations for certain variables or instances.

By one approach, the aforementioned input items can comprise at least one, two, three, or each of a patient image (such as, for example, computed tomography imagery and/or any imagery that comprises Digital Imaging and Communications in Medicine (DICOM)-compatible imagery (DICOM being a standard for managing, storing, and exchanging medical images and associated patient information)), a fluence map (where fluence represents radiative flux integrated over time and comprises a fundamental metric in dosimetry (i.e., the measurement and calculation of an absorbed dose of ionizing radiation in matter and tissue)), radiation treatment platform geometry information (including, for example, gantry angles, couch positions, collimator angles, and so forth), and target dose volume information (where, by one approach, the latter comprises sparsely-read target dose volume information).

If desired, each of the aforementioned plurality of different resultant radiation doses that comprise the training corpus can correspond to at least two of the aforementioned input items. As one illustrative example in those regards, these teachings will accommodate having at least two such input items comprise a patient image and target dose volume information.

At block 202, this process 200 then provides for training the aforementioned neural network using the above-described training corpus. By one approach, such training can be performed using gradient descent or other optimization objects by minimizing a loss function such as L1 between the output dose volume and a target dose volume from a gold standard dose engine. Curriculum learning can also be employed if desired by initially simplifying the dose calculation problem and then subsequently increasing difficulty until the original problem difficulty is reached. In this case, simplifying the problem can be achieved by applying a low-pass filter such as a gaussian filter to the target dose volume. A larger sigma of the filter corresponds to a simpler problem to solve. Similar simplification could also be achieved by performing spatial averaging of values before calculating the loss.

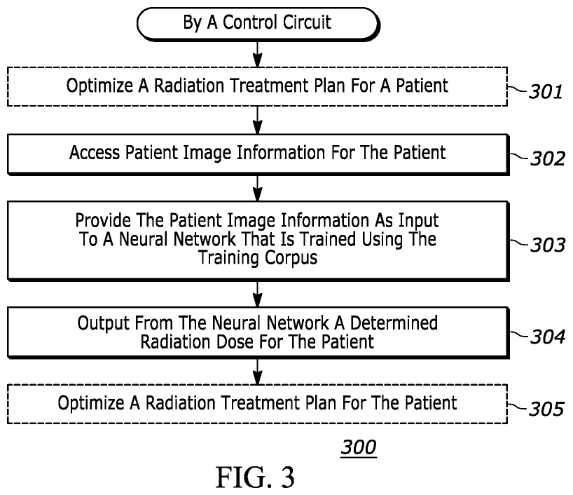

FIG. 3 presents an illustrative example of using the resultant trained neural network. Optional block 301 illustrates that this process 300 can be carried out, if desired, subsequent to optimizing a radiation treatment plan for a given patient at block 301. In this case, the resultant information determined by this process 300 can be used, for example, to assess the quality of that optimized radiation treatment plan.

At block 302 this process provides for accessing patient image information for the patient. These teachings will accommodate a wide range of patient image information with computed tomography images likely being a useful example in many application settings. These teachings will also accommodate accessing patient image information for a plurality of different positions (i.e., fields of view) that, for example, depict one or more volumes of interest from various respective angles. By one approach, the control circuit 101 accomplishes accessing the patient image information by having the neural network select a read location from, for example, the aforementioned memory 102.

At block 303, this process 300 provides the accessed patient image information as input to the above-described trained neural network.

At block 304, the trained neural network processes the accessed patient image information and responsively outputs a determined radiation dose for the patient. By one approach, the trained neural network outputs this information, at least in part, by selecting a write location.

At optional block 305, that determined radiation dose for the patient can be employed in an ordinary manner while optimizing a radiation treatment plan for this patient.

Further details that comport with these teachings will now be presented. It will be understood that the specific details of these examples are intended to serve an illustrative purpose and are not intended to suggest any particular limitations with respect to these teachings.

By one approach, the deep learning algorithms employed in the above-described neural network can operate in an Image-To-Image (I2I) framework. Such an image can be, for example, an original (or resampled) DICOM image from a simulation computed tomography image or a perspective transformed image to a beams-eye-view (BEV). If desired, the algorithm(s) can operate on individual beamlets or on whole fluence maps together. One advantage of operating on individual beamlets is that the final dose can be composed of a sum of individual result doses, thus preserving the linearity property of dose computation. Linearity can also be a beneficial property when optimizing fluence maps. That said, individual beamlets may be wasteful in an I2I framework, at least because each beam reads a dense input volume and writes a dense output volume. The inputs and output sizes should not be too small because dose deposition can have long range effects that need to be captured.

It may be noted that transforming to a beams-eye-view can be a time-consuming process. Also, the dose deposition for a single beamlet will be mostly zero in most of the space captured by a simulation computed tomography image. Therefore, the applicant has determined that it can be wasteful (of both processing time and computational and storage resources) to always read and write dense/large input/output volumes in such an application setting.

Accordingly, these teachings will accommodate using sparse reads and writes from and to a volume for computing dose. Furthermore, the control circuit 101 itself can be allowed to decide where to read input values and where to write output dose. In particular, the control circuit 101 can generate new read and write operations based on previous read values and written values. Read and write operations can be sparse, meaning that the network specifies a set of coordinates at which to read or write. This offers an advantage compared to dense updates. Furthermore, reads and writes can have access to the whole DICOM defined space and are therefore not limited in the size of the spatial context around the beam in question. The latter benefit is achieved without needing to accommodate large/dense inputs and outputs.

By one approach, instead of the input to the control circuit 101 being an image, the control circuit can receive information of the queried beam as geometric information. This information can, for example, be the source position, ray direction, and ray size at the isocenter. Further information could include source spectral information. Alternatively, the relevant information could be modeled into the control circuit 101, making the approach specific for a particular source spectrum or the like. In addition, or instead of geometric information, a sequence of three-dimensional locations along the beam inside the DICOM space can be supplied as input.

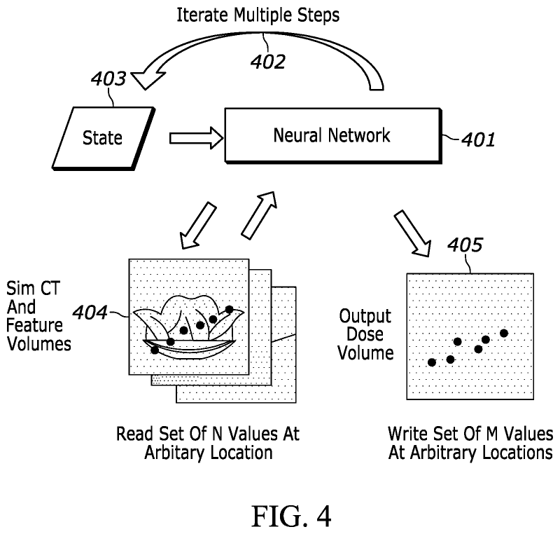

With reference to FIG. 4, the neural network 401 can be run in multiple steps 402, where steps may or may not share network weights and architecture. At each step the neural network 401 attends to the input producing a current state 403. This state 403 is then decoded to multiple read locations, multiple write locations and associated write values for the current step. Read locations are used to interpolate an input three-dimensional volume 404 that can contain the simulation computed tomography input as well as other channels that can encode additional spatial features. Such spatial features can be, for example, 1/(d*d) at every voxel where d is the distance between the voxel and the radiation source.

The read process can be based on volume interpolation using, for example, trilinear or spline interpolation. For at least some application settings, it can be useful that the volume interpolation operation has a well-defined gradient with respect to read location. The read operation can return a potentially multi-channel interpolated value for each location. By one approach, the read location can also interpolate the output dose volume 405 at the location and return this current dose value as an additional read channel. Read values and current state can then be encoded/combined into a new state.

Write locations and write values can be fed into write operations that add dose to an output dose volume. One way of writing the value to the output is to center a three-dimensional normal distribution point spread function at the write location and add values that are scaled with the write value. As before, a well-defined gradient with respect to the write location and write value can be useful for training.

By one approach, read and write locations that result from the network state can be added to locations along the beam axis. So configured, read and write locations that the neural network generates are then relative to the beam axis.

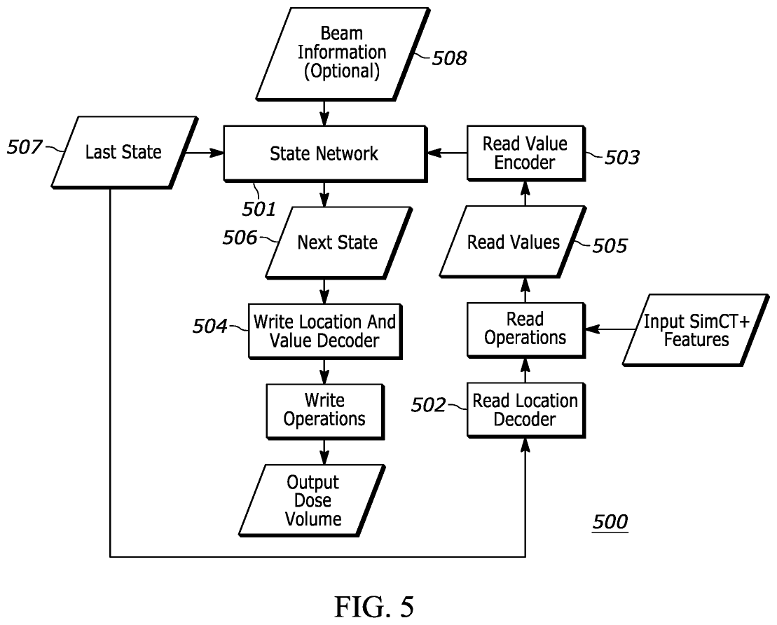

As one illustrative example in accord with these teachings, FIG. 5 presents one iteration of an inference phase 500 of a corresponding system. The State Network 501, the Read Location Decoder 502, the Read Value Encoder 503, and the Write Location Decoder 504 can be implemented using neural networks. (It will be understood that such elements as the Read Value Encoder 503 can be optional if the State Network 501 directly receives read values 505 as an input. After each iteration, the next state 506 becomes the last state 507.

By one illustrative approach, a fixed set of read locations along the beam can be generated, read, and supplied to the network as initial beam information 508. By one approach, such positions can be evenly spaced along the beam.

By one approach, the actual level of flux does not need to be an input to the networks itself. Instead, the dose that is written can be multiplied by the flux level to help ensure proportionality. Dose distributions for multiple beams or a from a two-dimensional fluence map can be generated by summing individual beam doses.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

What is claimed is:

1. A method of training a neural network for radiation dose determination, the method comprising:
accessing a training corpus comprising:
a plurality of different resultant radiation doses; and
a plurality of different input items that each correspond to a particular one of the plurality of different resultant radiation doses, wherein the input items comprise at least one of:
a patient image;
a fluence map;
radiation treatment platform geometry information; or
target dose volume information; and
training the neural network using the training corpus.

2. The method of claim 1, wherein the neural network comprises a transformer neural network.

3. The method of claim 1, wherein the target dose volume information comprises sparsely-read target dose volume information.

4. The method of claim 3, wherein at least some of the plurality of different resultant radiation doses each comprises sparsely-written resultant radiation doses.

5. The method of claim 1, wherein each of the plurality of different resultant radiation doses corresponds to at least two of a patient image, a fluence map, radiation treatment platform geometry information, and target dose volume information.

6. The method of claim 1, wherein each of the plurality of different resultant radiation doses corresponds to at least three of a patient image, a fluence map, radiation treatment platform geometry information, and target dose volume information.

7. The method of claim 1, wherein each of the plurality of different resultant radiation doses corresponds to each of a patient image, a fluence map, radiation treatment platform geometry information, and target dose volume information.

8. The method of claim 1, wherein the patient image comprises computed tomography imagery.

9. The method of claim 1, wherein the patient image comprises Digital Imaging and Communications in Medicine-compatible imagery.

10. A method of determining a radiation dose for a patient, the method comprising:
accessing patient image information for the patient;
providing the patient image information as input to a neural network that is trained using a training corpus that comprises:
a plurality of different resultant radiation doses; and
a plurality of different input items that each correspond to a particular one of the plurality of different resultant radiation doses, wherein the input items comprise at least one of:
a patient image;
a fluence map;
radiation treatment platform geometry information; or
target dose volume information; and
outputting from the neural network a determined radiation dose for the patient.

11. The method of claim 10 wherein each of the plurality of different resultant radiation doses that comprise the training corpus corresponds to at least two of the input items.

12. The method of claim 11 wherein the at least two of the input items comprise a patient image and target dose volume information.

13. The method of claim 10 wherein the target dose volume information comprises sparsely-read target dose volume information.

14. The method of claim 10 wherein the patient image comprises computed tomography imagery.

15. The method of claim 10, wherein the neural network comprises a transformer neural network.

16. The method of claim 10, wherein outputting the determined radiation dose for the patient occurs prior to optimizing a radiation treatment plan for the patient.

17. The method of claim 10, wherein outputting the determined radiation dose for the patient occurs subsequent to optimizing a radiation treatment plan for the patient.

18. The method of claim 10, wherein providing the patient image information as input to a neural network comprises the neural network selecting a read location.

19. The method of claim 10 wherein outputting from the neural network a determined radiation dose for the patient comprises the neural network selecting a write location.

20. The method of claim 10 wherein:
providing the patient image information as input to the neural network comprises providing patient image information for a plurality of different positions.
